(19) United States
(12) Patent Application Publication
(10) Pub. No.: US 2011/0245615 A1
(43) Pub. Date: Oct. 6, 2011

(54) BENDING-INSERTION ASSISTING INSTRUMENT AND INSERTION PATH SECURING APPARATUS

(75) Inventor: Masayuki IWASAKA, Kanagawa (JP)

(73) Assignee: FUJIFILM CORPORATION, Tokyo (JP)

(21) Appl. No.: 13/017,357

(22) Filed: Jan. 31, 2011

(30) Foreign Application Priority Data
Mar. 31, 2010 (JP) 2010-084417

(51) Int. Cl. A61B 1/00

(52) U.S. Cl. 600/146

(57) ABSTRACT

An insertion path securing apparatus includes a bending-insertion assisting instrument and a sheath tube that includes a first lumen where the bending-insertion assisting instrument is inserted. The bending-insertion assisting instrument includes a bendable tip bending portion, an intermediate extension portion being connected to the tip bending portion, and a bending operation portion being disposed at a base end of the intermediate extension portion. The state of the intermediate extension portion can be selectively switched to a variable-shape state where the bent shape of the intermediate extension portion can be freely changed and a shape retaining state where the bent shape of the intermediate extension portion is retained. The sheath tube includes a tube-like insertion portion of which the state can be selectively switched to a variable-shape state and a shape retaining state, and an operation portion that is disposed at a base end of the tube-like insertion portion.

### CROSS-REFERENCE TO RELATED APPLICATIONS

This application claims the benefit of Japanese Patent Application No. 2010-084417, filed on Mar. 31, 2010, the entire contents of which are hereby incorporated by reference, the same as if set forth at length; the entire of which are incorporated herein by reference.

### BACKGROUND OF THE INVENTION

1. Technical Field

The present invention relates to a bending-insertion assisting instrument and an insertion path securing apparatus using the bending-insertion assisting instrument.

2. Description of Related Art

When a treatment or an observation is to be made in a body cavity with various medical instruments such as an endoscope, a tube-like sheath tube (overtube) having flexibility is inserted into the body cavity and various medical instruments are inserted into the sheath tube, so that an insertion path into the body cavity is secured. Further, a sheath tube is also used to form a small hole by the incision of the wall of the body cavity or the skin and to perform the treatment of a specific portion in the body cavity with low invasiveness. As this kind of sheath tube, there is a sheath tube that can select a flexible variable-shape state and a state where the shape is retained and fixed (for example, US 2008/0039691).

Further, there is also provided a sheath tube having a double-tube structure where two sheath tubes capable of selecting a variable-shape state and a fixed state are superimposed (JP-A-2005-046275). According to this sheath tube, if the other sheath tube, which is in the variable-shape state, is moved relative to one sheath tube that is in the fixed state, it may be possible to make the sheath tubes alternately enter the insertion path and to insert the endoscope so that the endoscope follows the shape of the inside of the body cavity in the inner sheath tube.

According to the above-mentioned sheath tube, it may be possible to easily insert various medical instruments into the body cavity along an appropriate path. However, if an insertion path to be set has a complicatedly bent shape, it is difficult to continue to retain the shape of each bending position of the insertion path as it is. That is, it is possible to form a desired bent shape at the foremost portion of the sheath tube inserted into the body cavity. However, at a portion of the sheath tube that is closer to the base end than the foremost portion and which has already been bent, it is not possible to retain the bent shape, which has been formed once, as it is. Accordingly, the bent shape is changed while the sheath tube is being inserted. Further, in the sheath tube disclosed in JP-A-2005-046275, if the alternate retention of the shape of the inner and outer sheath tubes is repeated, the bent shape is gradually released, so that the initial bent shape is lost. Furthermore, neither of the structures of the sheath tubes is an excellent structure for reducing a diameter, and the diameters thereof are further increased due to the diameter of the endoscope.

### SUMMARY

An object of the invention is to provide a bending-insertion assisting instrument that can make various pieces of medical equipment reliably follow the bent shape of an insertion path and always accurately maintain the insertion path of the various pieces of medical equipment in a desired shape even though the insertion path along which the various pieces of medical equipment are inserted into the body cavity has a complicated shape, and an insertion path securing apparatus using the bending-insertion assisting instrument. Further, another object of the invention is to reduce the diameter of an insertion path securing apparatus.

The invention has the following structure.

(1) A bending-insertion assisting instrument bends an insertion path of a medical instrument being to be inserted into the body cavity in a desired shape so that the medical instrument is guided in the body cavity. The bending-insertion assisting instrument includes a tip bending portion, an intermediate extension portion, a bending operation portion and a shape switching operation portion. The tip bending portion includes a bendable bending mechanism. The intermediate extension portion has a long shape and is connected to the tip bending portion. The bending operation portion is disposed at a base end of the intermediate extension portion and bends the tip bending portion. The intermediate extension portion includes a shape retaining mechanism which selectively switches a state of the intermediate extension portion to a variable-shape state where the bent shape of the intermediate extension portion is freely changed and a shape retaining state where the bent shape of the intermediate extension portion is retained. The shape switching operation portion switches the respective states of the intermediate extension portion.

(2) An insertion path securing apparatus includes a bending-insertion assisting instrument and a sheath tube. The sheath tube includes a first lumen where a tip bending portion and an intermediate extension portion of the bending-insertion assisting instrument are inserted. The sheath tube includes a tube-like insertion portion and an operation portion. The tube-like insertion portion has a long shape and is to be inserted into the body cavity. The operation portion is disposed at a base end of the tube-like insertion portion. The tube-like insertion portion includes a shape retaining mechanism which selectively switches a state of the tube-like insertion portion to a variable-shape state where the bent shape of the tube-like insertion portion is freely changed and a shape retaining state where the bent shape of the tube-like insertion portion is retained. The operation portion switches the respective states of the tube-like insertion portion.

(3) A bending-insertion assisting instrument includes a tip bending portion, an intermediate extension portion and a device operating portion. The tip bending portion includes a bendable bending mechanism. The intermediate extension portion has a long shape and is connected to a base end of the tip bending portion. The intermediate extension portion includes a shape retaining mechanism which selectively switches a state of the intermediate extension portion to a variable-shape state where the bent shape of the intermediate extension portion is freely changed and a shape retaining state where the bent shape of the intermediate extension portion is retained. The device operating portion includes a bending operation portion and a shape switching operation portion. The bending operation portion is disposed at a base end of the intermediate extension portion and performs an operation for bending the tip bending portion. The shape switching operation portion switches the respective states of the intermediate extension portion. The bending operation portion and the shape switching operation portion are independently operated. The bending operation portion performs the operation for bending the tip bending portion without changing the bent shape of the intermediate extension portion.

According to the bending-insertion assisting instrument and the insertion path securing apparatus of the invention, it may be possible to reliably secure an insertion path having a desired shape while reliably retaining the shape of the sheath tube having been bent once.

### BRIEF DESCRIPTION OF THE DRAWINGS

FIG. 1 is a view illustrating an embodiment of the invention, and is a view showing the entire configuration of an insertion path securing apparatus.

FIG. 2 is a view showing the appearance of a bending-insertion assisting instrument.

FIG. 3 is a sectional view schematically showing a cross-section taken along a line A-A of FIG. 2.

FIG. 4 is a view showing the appearance of a sheath tube.

FIGS. 5A to 5E are views sequentially illustrating a first insertion path securing sequence that uses the insertion path securing apparatus.

FIG. 6 is a view showing the bent shape of the insertion path securing apparatus that is inserted into a large intestine as an example of the use of the insertion path securing apparatus.

FIG. 7 is a view showing the bent shape of the insertion path securing apparatus that is used for a transvaginal procedure as another example of the use of the insertion path securing apparatus.

FIGS. 8A to 8E are views sequentially illustrating a second insertion path securing sequence that uses the insertion path securing apparatus.

FIG. 9A is a view showing the structure of main parts of a modification of a shape retaining mechanism of the sheath tube, and FIGS. 9B and 9C are sectional views showing that joint rings are joined to each other.

FIG. 10 is a view showing the appearance of a modification of a tip bending portion of the bending-insertion assisting instrument.

FIGS. 11A and 11B are views illustrating a third insertion path securing sequence that uses an insertion path securing apparatus including a sheath tube and a bending-insertion assisting instrument of a modification shown in FIG. 10.

FIG. 12 is a view showing the appearance of a first modification of the insertion path securing apparatus.

FIG. 13 is a view showing the appearance of a second modification of the insertion path securing apparatus.

FIG. 14 is a view showing the appearance of a third modification of the insertion path securing apparatus.

FIG. 15 is a view showing the appearance of a fourth modification of the insertion path securing apparatus.

FIG. 16A is a schematic sectional view of a tip bending portion of a bending-insertion assisting instrument that is formed of a pneumatic actuator and FIG. 16B is a schematic cross-sectional view of the tip bending portion.

FIG. 17A is a schematic sectional view of a tip bending portion of a bending-insertion assisting instrument that is formed of a pneumatic actuator and FIG. 17B is a schematic cross-sectional view of the tip bending portion.

FIG. 18 is a schematic sectional view of a tip bending portion of a bending-insertion assisting instrument where a shape memory alloy is partially disposed.

FIG. 19A is a schematic sectional view showing a linear state of the tip bending portion shown in FIG. 18 and FIG. 19B is a schematic sectional view showing a bent state of the tip bending portion.

### DETAILED DESCRIPTION OF EMBODIMENTS OF THE INVENTION

An embodiment of the invention will be described in detail below with reference to the drawings.

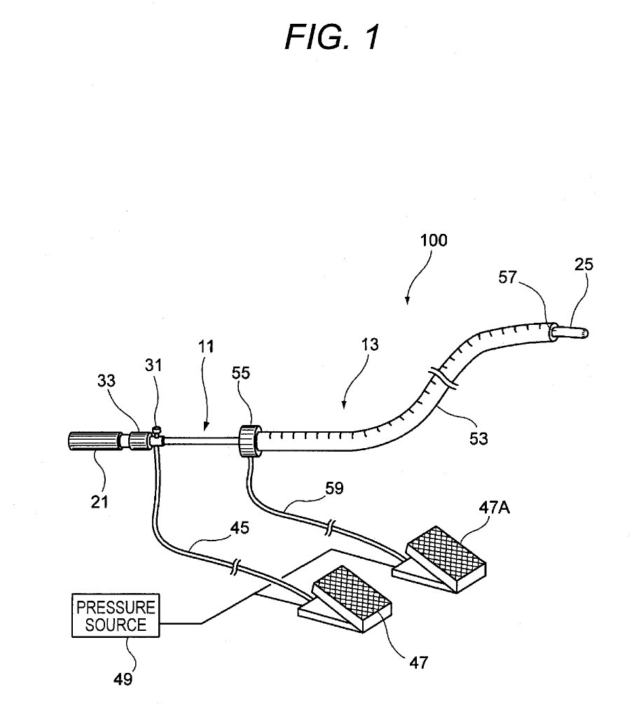

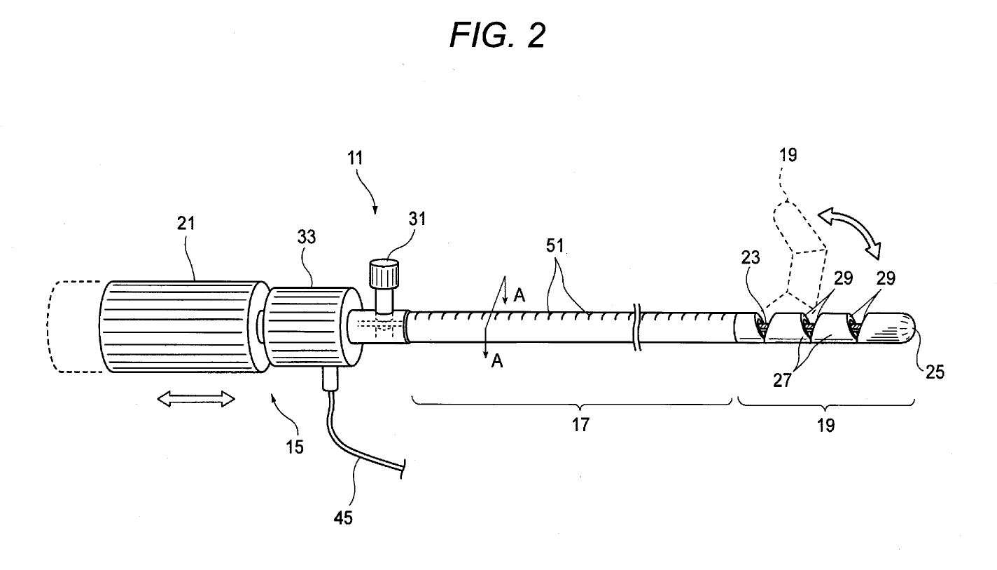

FIG. 1 is a view illustrating an embodiment of the invention, and is a view showing the entire configuration of an insertion path securing apparatus. FIG. 2 is a view showing the appearance of a bending-insertion assisting instrument. FIG. 3 is a sectional view schematically showing a cross-section taken along a line A-A of FIG. 2.

As shown in FIG. 1, an insertion path securing apparatus 100 is to secure a bent insertion path in a desired shape when medical instruments (various medical instruments such as an endoscope and a treatment instrument) are inserted into the body cavity. The insertion path securing apparatus 100 includes a bending-insertion assisting instrument 11 and a long tube-like sheath tube 13.

Referring to FIGS. 2 and 3, the bending-insertion assisting instrument 11 includes a bending operation portion 15, a long intermediate extension portion 17, a tip bending portion 19, and a traction wire 23. The long intermediate extension portion 17 is connected to the bending operation portion 15. The tip bending portion 19 extends from the intermediate extension portion 17 and includes a bending mechanism formed at the tip thereof. The traction wire 23 is inserted through the intermediate extension portion 17 and the tip bending portion 19. One end of the traction wire 23 is fixed to the tip portion of the tip bending portion 19, and the other end of the traction wire 23 is fixed to an operation handle (bending operation portion) 21 of the bending operation portion 15.

A tip piece 25 and a plurality of bending pieces 27, which form the bending mechanism, are provided at the tip bending portion 19, and the end faces of the respective pieces facing each other have notched portions 29 that are obliquely cut off. The respective pieces 25 and 27 are connected to each other so that the notched portions 29 have the same side positions relative to the central axis of the tip bending portion 19. Further, a fixing screw 31, which fixes the traction wire 23 in the intermediate extension portion 17 by pressing the traction wire 23, is provided at the base end portion of the intermediate extension portion 17 facing the bending operation portion 15. That is, the bending operation portion 15 is operated to bend the tip bending portion 19, and a foot switch 47 is a shape switching operation portion for switching the state of the intermediate extension portion. The bending operation portion 15 and the foot switch 47 function as device operating portions that can be independently operated, respectively.

Accordingly, when the operation handle 21 of the bending operation portion 15 is operated to recede from a fixed portion 33, the traction wire 23 is pulled from the tip bending portion 19 and the end face of the tip bending portion 19 is operated to be bent until the tip piece 25 comes into contact with the end face of the notched portion 29 of the bending piece 27. If the fixing screw 31 is tightened, the traction wire 23 is fixed. Accordingly, the bent state of the tip bending portion 19 can be maintained. Further, if the fixing screw 31 is loosened, the bent state of the tip bending portion 19 is released. Furthermore, gradations 51 are formed on the outer peripheral surface of the intermediate extension portion 17 in the longitudinal direction. Accordingly, it may be possible to find out the insertion length of the intermediate extension portion 17, that is, the insertion position in the sheath tube 13 in the longitudinal direction. Meanwhile, even though the operation handle 21 is operated to advance or retract to or from the fixed portion 33, the bent shape of the intermediate extension portion 17 is not changed.

As shown in FIG. 3, the intermediate extension portion 17 includes an inner sleeve 35 and an outer sleeve 37. The outer sleeve 37 is disposed to receive the inner sleeve 35 with a hollow space 39 interposed between itself and the outer peripheral surface of the inner sleeve 35, and hardly elongates and contracts in the longitudinal direction. A plurality of protrusions 41 and 43, which protrudes in the radial direction so as to face each other, is formed on the outer peripheral surface of the inner sleeve 35 and the inner peripheral surface of the outer sleeve 37. Both the end portions of the inner sleeve 35 and the outer sleeve 37, which face the tip bending portion 19 and the fixed portion 33, are airtightly fixed. Further, a center hole 35a through which the traction wire 23 passes is formed at the center of the inner sleeve 35.

A fluid supply pipe 45, which communicates with the hollow space 39, is connected to the fixed portion 33. The fluid supply pipe 45 is connected to a fluid supply source 49 through the foot switch 47 that is the shape switching operation portion (see FIG. 1). The foot switch 47 is operated, so that fluid is supplied to the hollow space 39 from the fluid supply source 49 or discharged from the hollow space 39.

If fluid is discharged from the hollow space 39, the outer sleeve 37 thinner than the inner sleeve 35 is deformed so that the diameter of the outer sleeve 37 is reduced. Accordingly, the protrusions 41 and 43 are engaged with each other and the bending stiffness of the intermediate extension portion 17 is increased. Therefore, the intermediate extension portion 17 enters a shape retaining state where the bent shape of the intermediate extension portion 17 is retained. Further, if fluid is introduced to the hollow space 39, the engagement between the protrusions 41 and 43 is released. Accordingly, the intermediate extension portion 17 enters a variable-shape state where the bent shape of the intermediate extension portion 17 can be freely changed. That is, if the foot switch 47 is operated to be switched, the plurality of protrusions 41 and 43 of the inner and outer sleeves 35 and 37, which is a shape retaining mechanism, is engaged or disengaged with or from each other. Accordingly, the state of the intermediate extension portion 17 can be selectively switched to the shape retaining state where the bent shape of the intermediate extension portion 17 is retained as it is, and the variable-shape state where the bent shape of the intermediate extension portion 17 can be freely changed. Further, since the traction wire 23 passes through the center of the intermediate extension portion 17, the shape of the intermediate extension portion 17 can exert a stable shape retaining property without being affected even though tension is generated on the traction wire by an operation for bending the tip bending portion 19.

FIG. 4 is a view showing the appearance of the sheath tube. As shown in FIG. 4, the sheath tube 13 includes a long tube-like insertion portion 53 that is to be inserted into the body cavity and an operation portion 55 that is provided at the base end of the tube-like insertion portion 53. The tube-like insertion portion 53 includes a guide hole 57 that has openings at both ends thereof and is formed in the longitudinal direction, and the bending-insertion assisting instrument 11 or the like is removably inserted into the guide hole 57. That is, the tube-like insertion portion 53 functions as a lumen where the tip bending portion 19 and the intermediate extension portion 17 of the bending-insertion assisting instrument 11 are inserted. Further, when an endoscopic insertion portion of an endoscope is used while being inserted into the tube-like insertion portion 53 of the sheath tube 13, it is preferable that at least the tip portion to be inserted into the body cavity be made of a translucent material having flexibility, such as an acrylic resin, a polyethylene resin, or a polyvinyl chloride, for the easy observation using the endoscope.

The tube-like insertion portion 53 of the sheath tube 13 includes a shape retaining mechanism that has the same structure as the structure of the intermediate extension portion 17 of the bending-insertion assisting instrument 11 shown in FIG. 3. That is, the tube-like insertion portion 53 has a double-sleeve structure that includes an inner sleeve (not shown) and an outer sleeve receiving the inner sleeve with a hollow space interposed therebetween. A plurality of protrusions, which protrudes in the radial direction so as to face each other, is formed on the outer peripheral surface of the inner sleeve and the inner peripheral surface of the outer sleeve. A fluid supply pipe 59, which communicates with the hollow space, is connected to the operation portion 55 of the sheath tube 13. The fluid supply pipe 59 is connected to the fluid supply source 49 through a foot switch 47A that is a shape switching operation portion (see FIG. 1). The foot switch 47A is operated, so that fluid is supplied to the hollow space from the fluid supply source 49 or discharged from the hollow space. Accordingly, the state of the tube-like insertion portion 53 can be selectively switched to a variable-shape state where the bent shape of the tube-like insertion portion 53 can be freely changed and a shape retaining state where the bent shape of the tube-like insertion portion 53 is retained as it is.

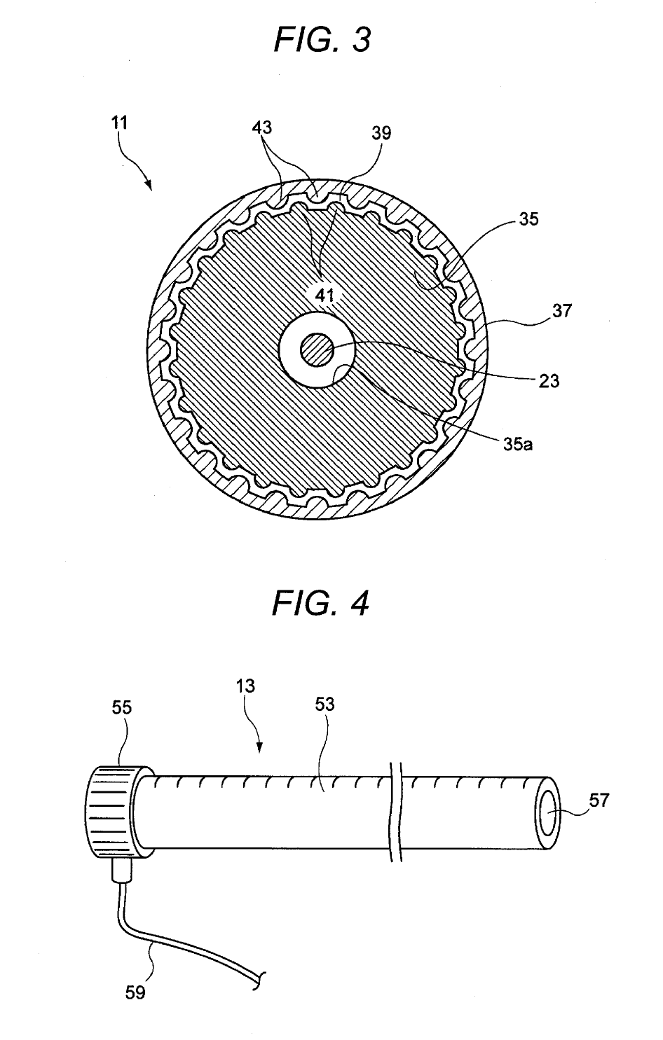

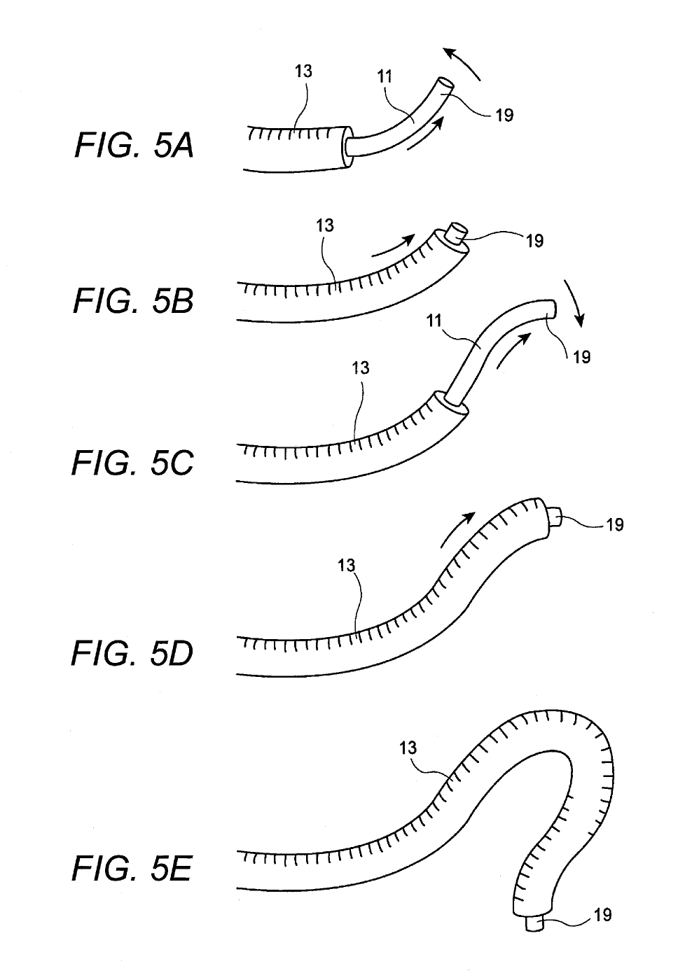

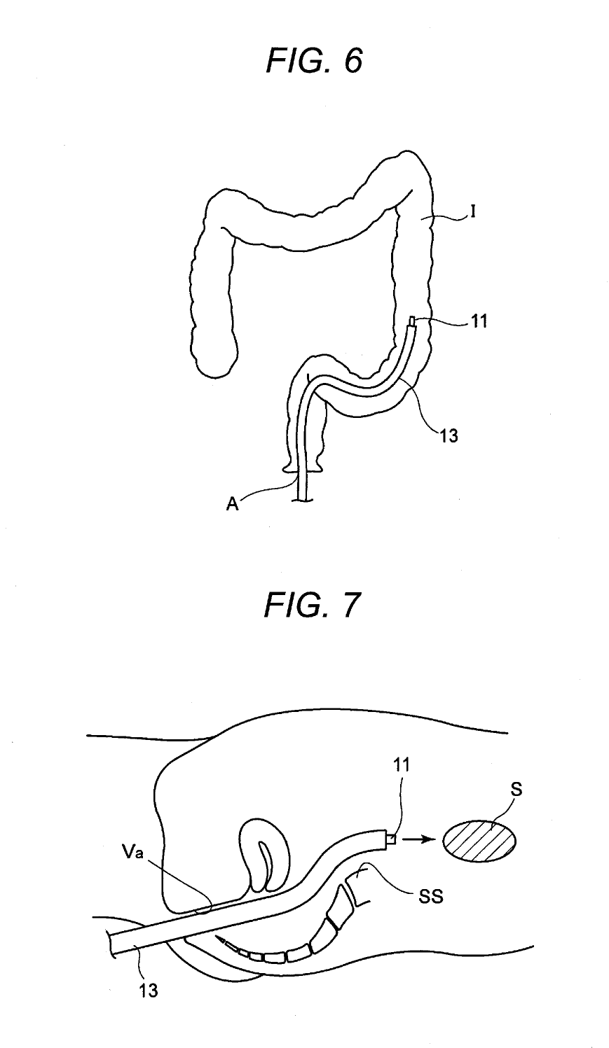

A first insertion path securing sequence will be described below with reference to FIGS. 5A to 5E, FIG. 7, and Table 1. In the first insertion path securing sequence, an insertion path for the insertion of a medical instrument such as an endoscope into the body cavity through a natural opening of a human body, such as a mouth, an anus, or a vagina, is secured by the insertion path securing apparatus 100 having the above-mentioned structure. FIGS. 5A to 5E are views sequentially illustrating a first insertion path securing sequence that uses an insertion path securing apparatus, FIG. 6 is a view showing the bent shape of the insertion path securing apparatus that is inserted into a large intestine as an example of the use of the insertion path securing apparatus, and FIG. 7 is a view showing the bent shape of the insertion path securing apparatus that is used for a transvaginal procedure as another example of the use of the insertion path securing apparatus.

As shown in FIGS. 5A to 5E and Table 1, first, in Step 1, the foot switch 47A is operated, so that fluid is discharged from the sheath tube 13 and the protrusions are engaged with each other and the sheath tube 13 enters the shape retaining state. Further, the foot switch 47 is operated, so that the engagement between the plurality of protrusions 41 and 43 of the inner and outer sleeves 35 and 37 of the bending-insertion assisting instrument 11 is released. Furthermore, after the fixing screw 31 is loosened to make the bending-insertion assisting instrument 11 enter the variable-shape state, the bending-insertion assisting instrument 11 having been in the variable-shape state is inserted into the sheath tube 13 while in the shape retaining state and protrudes from the tip of the sheath tube 13.

TABLE 1

| | | Bending-insertion assisting | | |
|---|---|---|---|---|
| | | instrument | | |
| | | Intermediate | | |
| | | extension | Tip bending | |
| | Operation | portion | portion | Sheath tube |
| Step 1 | Insertion of | Shape variable | Free | Shape retaining |
| | bending- | | | |
| | insertion | | | |
| | assisting | | | |
| | instrument | | | |
| Step 2 | Bending | Shape variable | Bent | Shape retaining |
| Step 3 | | Shape retaining | Bent | Shape variable |
| Step 4 | Insertion of | Shape retaining | Bent | Shape variable |
| | sheath tube | | | |
| Step 5 | | Shape variable | Free | Shape retaining |

In Step 2, the operation handle 21 of the bending-insertion assisting instrument 11 is operated to recede from the fixed portion 33 so that the traction wire 23 is pulled from the tip bending portion 19 and the tip bending portion 19 is bent in a desired shape. Then, the fixing screw 31 is tightened, so that the traction wire 23 is fixed and maintained in the bent state (FIG. 5A).

Then, in Step 3, after the intermediate extension portion 17 enters the shape retaining state so as to correspond to the shape of the sheath tube 13 while the bent shape of the tip of the bending-insertion assisting instrument 11 is retained, the sheath tube 13 enters the variable-shape state. In Step 4, the sheath tube 13 is guided in the bent shape of the bending-insertion assisting instrument 11 and is moved to the vicinity of the tip of the bending-insertion assisting instrument 11 (FIG. 5B). Accordingly, the bent shape of the bending-insertion assisting instrument 11 is transferred to the sheath tube 13.

Further, in Step 5, after the sheath tube 13 enters the shape retaining state, the first insertion path securing sequence returns to Step 1 and the bending-insertion assisting instrument 11 enters the variable-shape state and is inserted into the sheath tube 13. Then, the bending-insertion assisting instrument 11 protrudes from the tip of the sheath tube 13 (FIG. 5C).

After that, the same operations as described above are repeated. Accordingly, after being guided by the sheath tube 13 in the shape retaining state, protruding from the tip of the sheath tube 13, and being bent, the bending-insertion assisting instrument 11 having been put in the variable-shape state is put in the shape retaining state. The sheath tube 13 is guided by the bending-insertion assisting instrument 11 in the shape retaining state, enters the variable-shape state, and is moved to the vicinity of the tip of the bending-insertion assisting instrument 11. In this way, the states of the bending-insertion assisting instrument 11 and the sheath tube 13 are alternately changed into the shape retaining state and the variable-shape state, and the member (the sheath tube 13 or the bending-insertion assisting instrument 11) in the variable-shape state is moved while being guided by the member (the bending-insertion assisting instrument 11 or the sheath tube 13) in the shape retaining state. Accordingly, it may be possible to secure an insertion path in a desired shape by bending the sheath tube 13 in an arbitrary shape at an arbitrary position (FIGS. 5D and 5E).

According to the first insertion path securing sequence, even though an insertion path has a plurality of inflection points, the insertion path securing apparatus is sequentially inserted while the bent shape, which has already been formed, is reliably retained by any one of the sheath tube 13 and the intermediate extension portion 17 of the bending-insertion assisting instrument 11 that is in the shape retaining state. Accordingly, the bent shape is not lost. Further, the bent shape, which is newly formed by the tip bending portion 19 of the bending-insertion assisting instrument 11, is passed to the sheath tube 13 and then transferred to the intermediate extension portion 17 of the bending-insertion assisting instrument 11, so that the bent shape is reliably retained.

If the bending-insertion assisting instrument 11 is extracted from the sheath tube 13 after the insertion path is secured, it may be possible to easily insert or remove a medical instrument, such as an endoscope or a catheter, into or from the guide hole (lumen) 57 of the sheath tube 13 into which the bending-insertion assisting instrument 11 has been inserted and to increase the range of the procedure without burdening a patient.

As described above, the insertion path securing apparatus 100 according to this embodiment includes two members, that is, the bending-insertion assisting instrument 11 and the sheath tube 13. Accordingly, it may be possible to reduce the diameter of the insertion path securing apparatus 100.

FIG. 6 shows the bent shape of the insertion path securing apparatus when the insertion path securing apparatus 100 is inserted into a large intestine I from an anus A while being bent so as to correspond to the shape of the large intestine I according to the first insertion path securing sequence. FIG. 7 shows the bent shape of the insertion path securing apparatus when an S-shaped insertion path is secured from the vagina Va to the stomach S while avoiding the sacrum SS of a patient by a transvaginal procedure, for the removal of a stomach tumor. In any case, it may be possible to freely bend the insertion path so as to correspond to the state in the body cavity, and to reduce a burden on the patient.

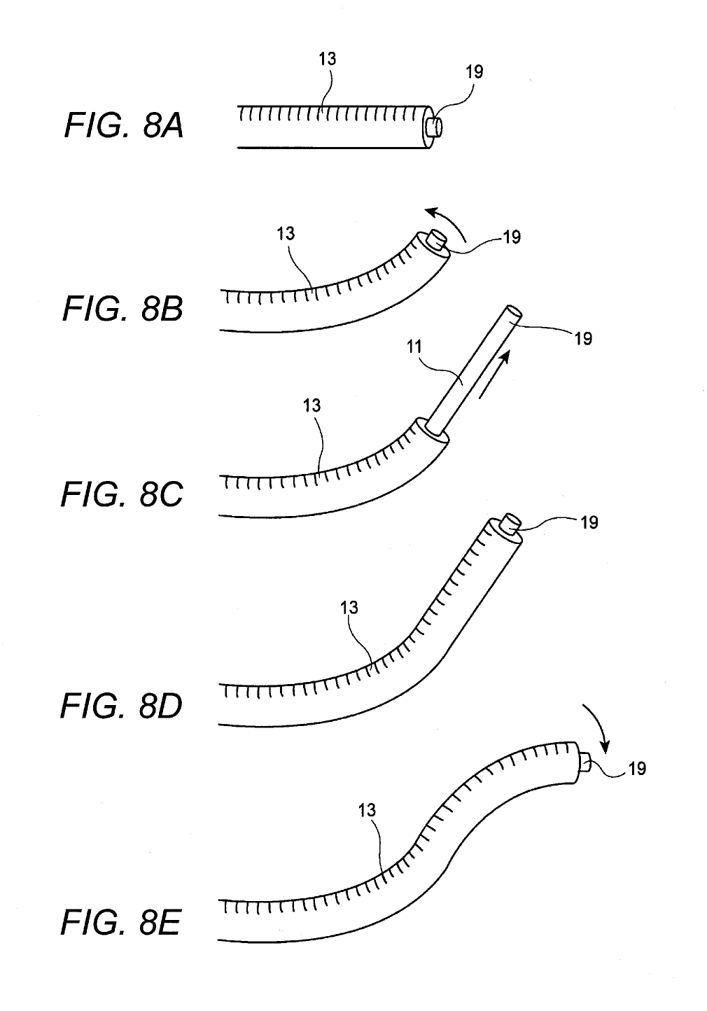

A second insertion path securing sequence for securing an insertion path will be described below with reference to FIGS. 8A to 8E and Table 2. FIGS. 8A to 8E are views sequentially illustrating a second insertion path securing sequence that uses an insertion path securing apparatus. Meanwhile, an insertion path securing apparatus 100 used in the second insertion path securing sequence is the same insertion path securing apparatus that is shown in FIG. 5 and used in the first insertion path securing sequence. The insertion path securing apparatus 100 used in the second insertion path securing sequence is different from that used in the first insertion path securing sequence only in the insertion sequence.

TABLE 2

| | | Bending-insertion assisting | | |
|---|---|---|---|---|
| | | instrument | | |
| | | Intermediate | | |
| | | extension | Tip bending | |
| | Operation | portion | portion | Sheath tube |
| Step 1 | Insertion of | Shape retaining | Free | Shape variable |
| | sheath tube | | | |
| Step 2 | Bending | Shape retaining | Bent | Shape variable |
| Step 3 | | Shape retaining | Bent | Shape retaining |
| Step 4 | Insertion of | Shape variable | Free | Shape retaining |
| | bending- | | | |
| | insertion | | | |
| | assisting | | | |
| | instrument | | | |
| Step 5 | | Shape retaining | Free | Shape retaining |

As shown in FIGS. 8A to 8E and Table 2, first, in Step 1 of the second insertion path securing sequence, the sheath tube 13 enters the variable-shape state, the intermediate extension portion 17 of the bending-insertion assisting instrument 11 enters the shape retaining state, and the sheath tube 13 is moved so as to correspond to the tip of the bending-insertion assisting instrument 11 while being guided by the bending-insertion assisting instrument 11 (FIG. 8A).

In Step 2, the tip bending portion 19 of the bending-insertion assisting instrument 11 is bent. Accordingly, as the bending-insertion assisting instrument 11 is bent, the sheath tube 13 in the variable-shape state is bent together with the bending-insertion assisting instrument 11 (FIG. 8B). Then, the sheath tube 13 enters the shape retaining state in Step 3 and the bending-insertion assisting instrument 11 enters the variable-shape state and protrudes from the tip of the sheath tube 13 in Step 4. After that, in Step 5, the intermediate extension portion 17 of the bending-insertion assisting instrument 11 enters the shape retaining state and is maintained while protruding (FIG. 8C).

Further, the second insertion path securing sequence returns to Step 1, and the sheath tube 13 enters the variable-shape state and is moved so as to correspond to the tip of the bending-insertion assisting instrument 11 (FIG. 8D). After that, the same operations as described above are repeated, so that it may be possible to bend the insertion path securing apparatus 100 in an arbitrary shape.

According to the second insertion path securing sequence, even though an insertion path has a plurality of inflection points, the bent shape of the insertion path securing apparatus is retained alternately by the sheath tube 13 and the bending-insertion assisting instrument 11 in the shape retaining state and the insertion path securing apparatus is sequentially inserted. Accordingly, the bent shape is not lost. Therefore, even though an insertion path has a complicated shape, it may be possible to simply and reliably form a path exactly following the insertion path and to retain the shape of a path once the path has been formed.

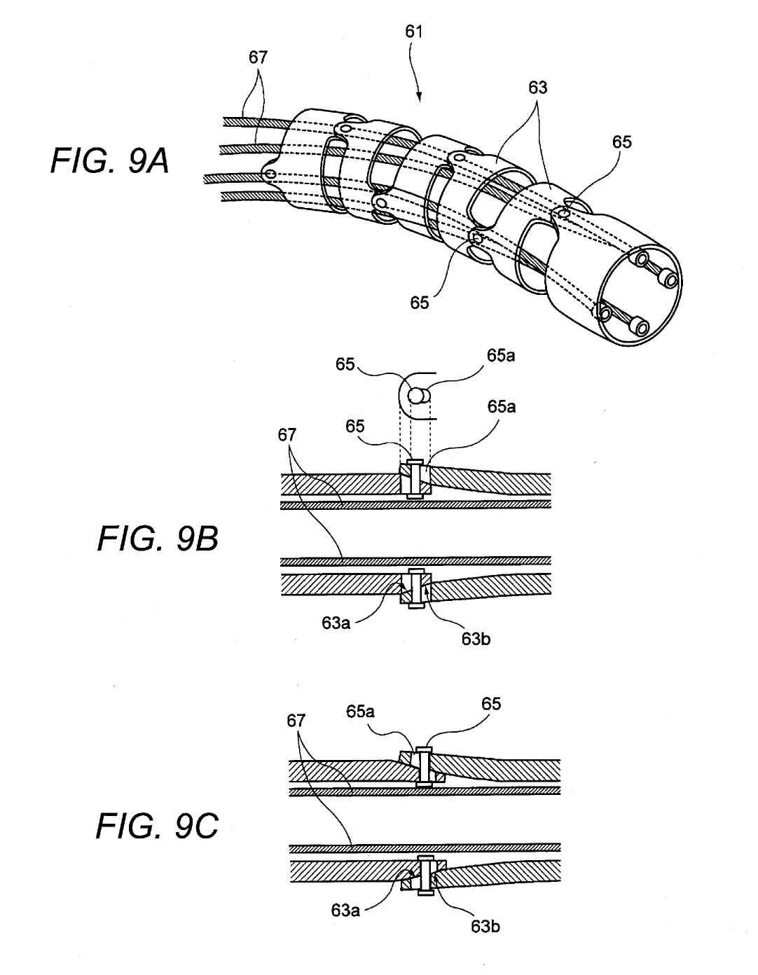

FIG. 9A is a view showing the structure of main parts of a modification of the shape retaining mechanism of the sheath tube. As shown in FIG. 9A, a sheath tube 61 of the modification includes a plurality of short cylindrical joint rings 63. The plurality of short cylindrical joint rings 63 is connected in series and respective adjacent joint rings 63 are connected to each other by connecting shafts 65 so as to be rotated relative to each other. The axial directions of the connecting shafts 65 have a phase difference of 90° therebetween. Accordingly, the plurality of joint rings 63 can be bent in an arbitrary direction. Further, four operation wires 67 are provided in the plurality of joint rings 63 so as to be parallel to the connecting direction of the joint rings 63. One end of each of the operation wires is fixed to the headmost joint ring 63, and the other end thereof is fixed to an operation handle of a bending operation portion (not shown).

Moreover, as shown in FIG. 9B, connection holes 65a through which the connecting shafts 65 pass are formed at the front and rear end portions of the joint ring 63, respectively. The connection hole 65a has a larger diameter in the connecting direction of the joint rings 63. Further, the joint ring 63 includes front slants 63a and rear slants 63b. Each of the front slants 63a is formed at a portion of the joint ring 63 including the front connection hole 65a, and the outer diameter of the front slant 63a is decreased toward the front end of the joint ring. Each of the rear slants 63b is formed at a portion of the joint ring 63 including the rear connection hole 65a, and the inner diameter of the rear slant 63b is increased toward the rear end of the joint ring. Here, the outer diameter of the front end portion of the joint ring 63 and the outer diameter of the rear portion of the front connection hole 65a are designed to be larger than the inner diameter of the rear end portion of the joint ring 63 and the inner diameter of the front portion of the rear connection hole 65a. Furthermore, if tension is generated on the operation wires 67, the front slant 63a of the joint ring 63 is frictionally engaged with the rear slant 63b of another joint ring 63 that is disposed on the front side as shown in FIG. 9C and the bent shape is retained. In this state, the bent shape is retained when the operation wire 67 is fixed by the fixing screw 31 that is provided at the bending operation portion, and the bent state is released when the fixing screw 31 is loosened.

The shape retaining mechanism of this modification, which includes the plurality of joint rings 63 and the plurality of operation wires 67, may be employed as the shape retaining mechanism of the bending-insertion assisting instrument 11.

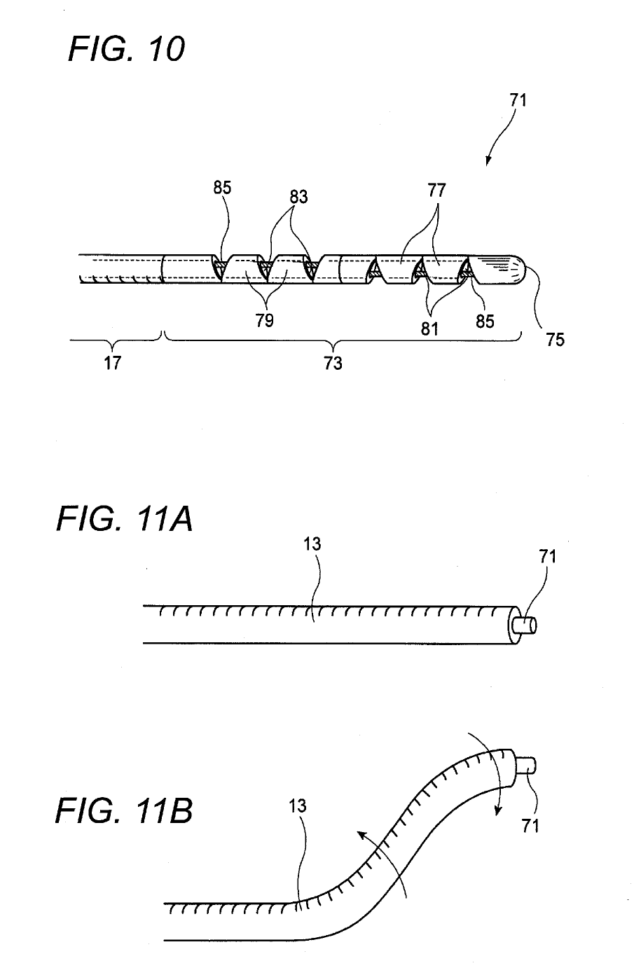

FIG. 10 is a view showing the appearance of a modification of a tip bending portion of the bending-insertion assisting instrument. As shown in FIG. 10, a tip piece 75 and a plurality of bending pieces 77 and 79 of a tip bending portion 73 of a bending-insertion assisting instrument 71 of the modification are connected to each other and form a bending mechanism. The end faces of the respective bending pieces 77 facing each other have notched portions 81 that are obliquely cut off. The end faces of the respective bending pieces 79 facing each other have notched portions 83 that are obliquely cut off. The bending pieces 77 and the bending pieces 79 are connected so as to have a phase difference of 90° therebetween with respect to the central axis of the tip bending portion 73. Further, one end of the bending-insertion assisting instrument 71 is fixed to the tip portion of the tip bending portion 73, and the other end thereof includes two traction wires 85 and 85 that are fixed to an operation handle (not shown). The respective traction wires 85 and 85 are disposed on the side to which the notched portions 81 and 83 of the plurality of bending pieces 77 and 79 are opened.

According to the bending-insertion assisting instrument 71 of this modification, it may be possible to bend the insertion path securing apparatus in different directions at the same time by appropriately pulling the two traction wires 85 and 85. Accordingly, as shown in FIGS. 11A and 11B, it may be possible to bend the bending-insertion assisting instrument 71 in different directions (in the S shape) at the same time while the bending-insertion assisting instrument 71 is inserted into the sheath tube 13.

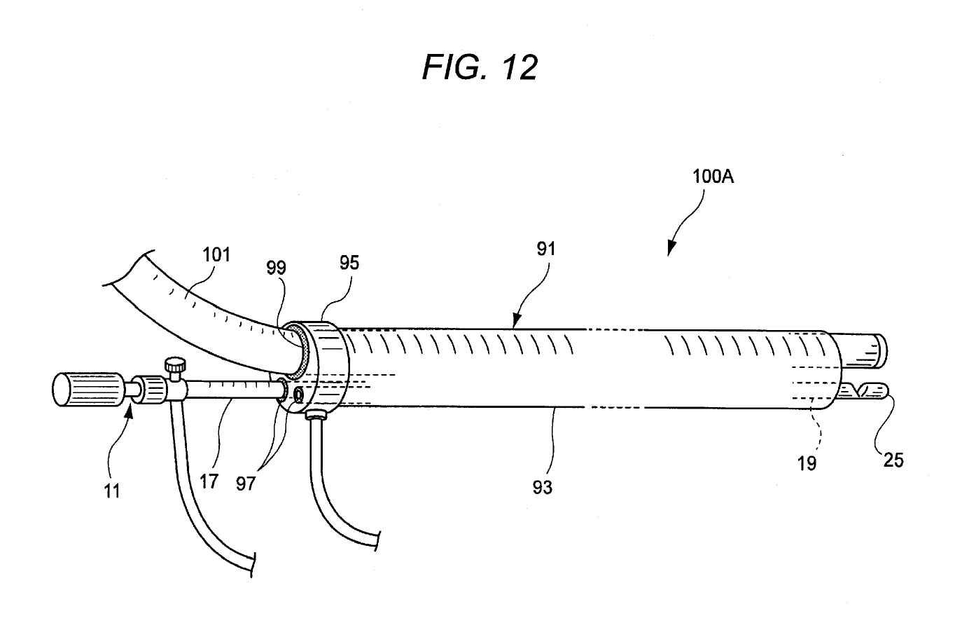

FIG. 12 is a view showing the appearance of a first modification of the insertion path securing apparatus. As shown in FIG. 12, an insertion path securing apparatus 100A of a first modification includes a bending-insertion assisting instrument 11 and a sheath tube 91. The sheath tube 91 includes a long tube-like insertion portion 93 that is to be inserted into the body cavity, and an operation portion 95 that is provided at the base end of the tube-like insertion portion 93. A guide hole 97 that is a first lumen into which the bending-insertion assisting instrument 11 is inserted and a guide hole 99 that is a second lumen into which an endoscopic insertion portion 101 to be inserted into the body cavity is inserted are formed at the tube-like insertion portion 93 so as to be opened to both ends of the tube-like insertion portion 93. According to the insertion path securing apparatus 100A of the first modification, the insertion path securing apparatus 100A of the first modification has the same effect as the effect of the insertion path securing apparatus 100 shown in FIG. 1, and has an advantage of operating the insertion path securing apparatus 100A while making an observation with an endoscope.

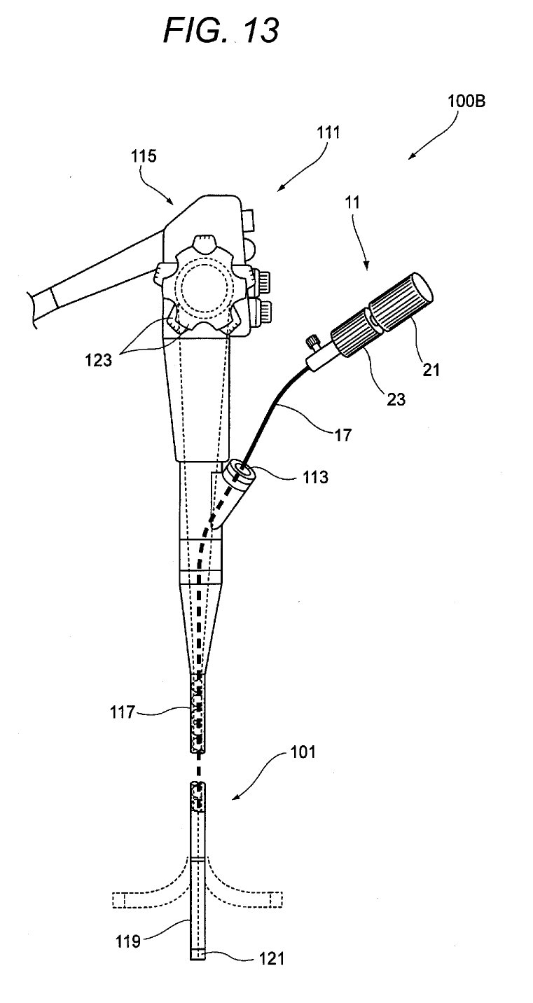

FIG. 13 is a view showing the appearance of a second modification of the insertion path securing apparatus. As shown in FIG. 13, an endoscopic insertion portion 101 of an endoscope 111 of an insertion path securing apparatus 100B of a second modification has a function of a sheath tube, and a bending-insertion assisting instrument 11 is inserted from the forceps insertion hole 113. That is, the endoscopic insertion portion 101 can retain the bent shape that is formed by the bending-insertion assisting instrument 11.

The endoscopic insertion portion 101 includes a flexible portion 117, a bending portion 119, and an endoscope tip portion 121 that are sequentially disposed from the side of a main body operating portion 115. As the structure shown in FIG. 3, the flexible portion 117, which can retain the bent shape, is provided with a shape retaining mechanism. The shape retaining mechanism includes an inner sleeve, and an outer sleeve that receives the inner sleeve with a hollow space interposed therebetween. Further, as in the endoscope 111 in the related art, an angle knob 123 of the main body operating portion 115 is rotated, so that the bending portion 119 is operated to be bent. According to the insertion path securing apparatus 100B having the above-mentioned structure, the endoscopic insertion portion 101 of the endoscope 111 serves as the above-mentioned sheath tube, has a bending function and a shape retaining function, and is operated with the combination of the bending function and the shape retaining function of the bending-insertion assisting instrument 11. Accordingly, it may be possible to freely secure an insertion path.

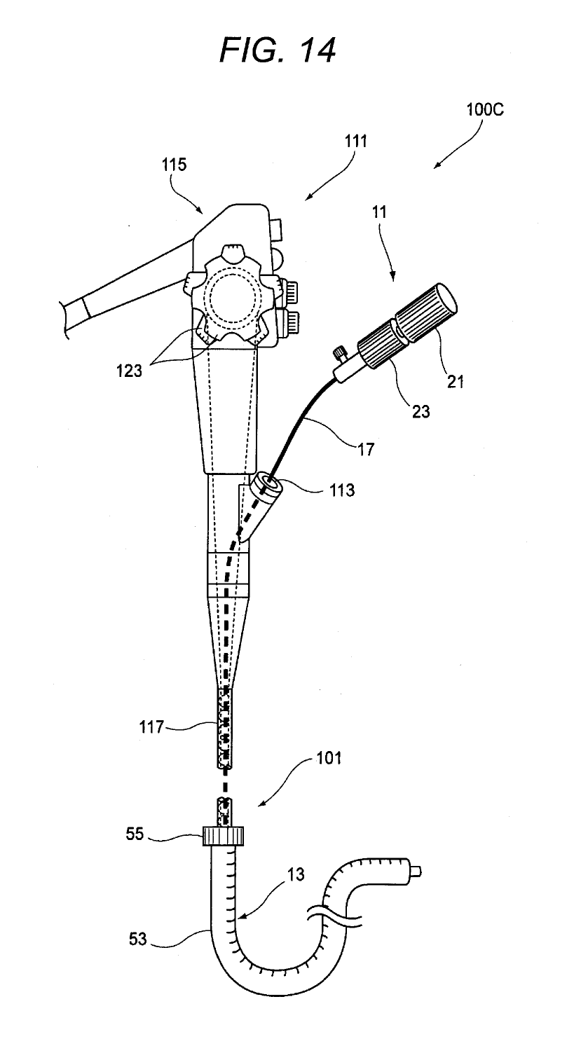

FIG. 14 is a view showing the appearance of a third modification of the insertion path securing apparatus. As shown in FIG. 14, an insertion path securing apparatus 100C of a third modification includes the endoscope 111 that is shown in FIG. 13, the bending-insertion assisting instrument 11 that has been described with reference to FIG. 2, and the sheath tube 13 that has been described with reference to FIG. 4. The endoscope 111 includes the flexible portion 117 of the endoscopic insertion portion 101 having a shape retaining function. According to the insertion path securing apparatus 100C having this structure, it may be possible to increase the number of devices having a bending function and a shape retaining function. Accordingly, it may be possible to selectively use the bending function and the shape retaining function of a device, which is the most easily operated, according to the procedure content or situation. As a result, it may be possible to smoothly perform a procedure.

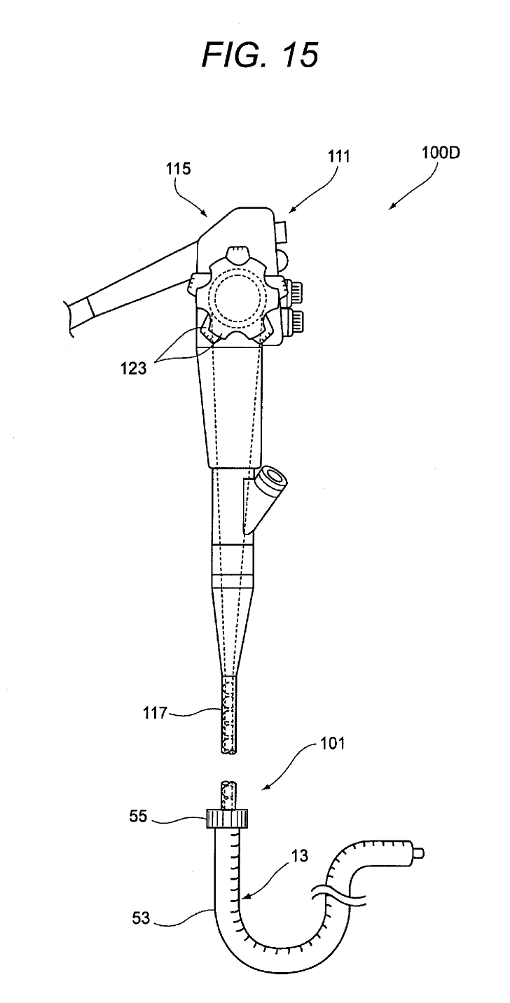

FIG. 15 is a view showing the appearance of a fourth modification of the insertion path securing apparatus. As shown in FIG. 15, an insertion path securing apparatus 100D of a fourth modification includes an endoscope 111 and the above-mentioned sheath tube 13. The endoscope 111 includes the endoscopic insertion portion having a shape retaining function. According to the insertion path securing apparatus 100D having this structure, only two devices are required. Accordingly, it is unnecessary to extract the bending-insertion assisting instrument 11 and insert an endoscope again after an insertion path is secured. Further, since it may be possible to make an observation with an endoscope during a bending operation, it may be possible to confirm whether obstacles exist in an observation field of view in a bending direction.

Meanwhile, it is preferable that the above-mentioned bending-insertion assisting instrument 11 or the endoscopes of the first to fourth modifications perform an operation for bending the tip bending portion 19 and the shape retaining operation of the intermediate extension portion 17 by separate and independent operation systems. Further, it is preferable that an operation for bending the tip bending portion 19 do not affect the shape of the intermediate extension portion 17. An operation for bending the tip bending portion has been performed using the traction wire 23 in the respective embodiments that have been exemplified. However, in this case, if the traction wire is guided by a guide member in the intermediate extension portion 17 so as to pass through a portion near the central axis regardless of the bent shape of the intermediate extension portion 17, the path of the traction wire 23 is the same regardless of the bent shape of the intermediate extension portion 17. Accordingly, the bent shape of the intermediate extension portion 17 is not affected.

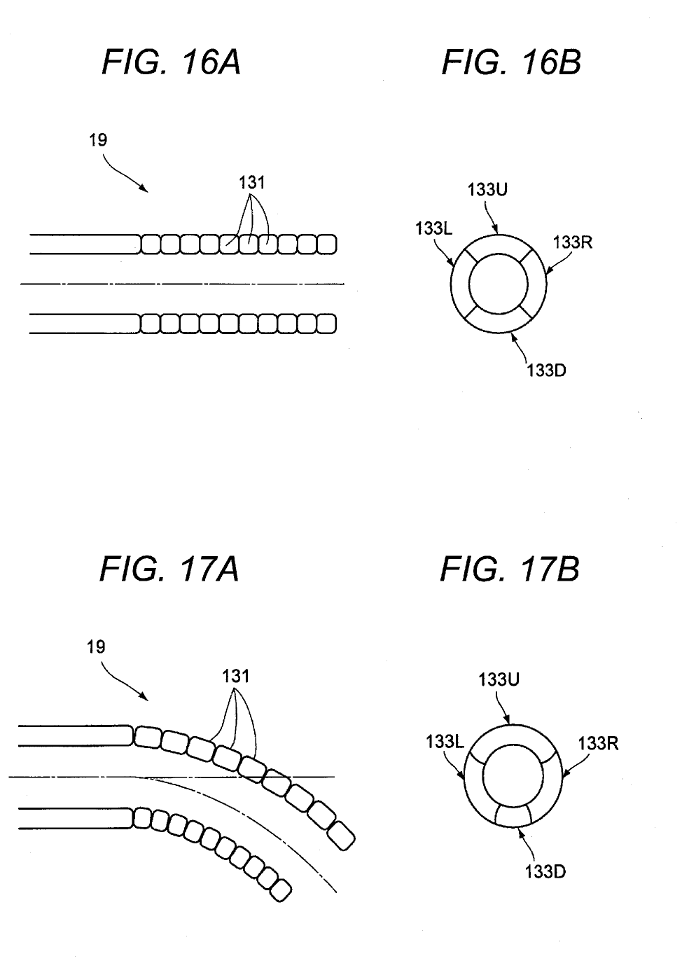

Further, the tip bending portion 19 is formed of a pneumatic actuator other than the traction wire 23 and a signal corresponding to air pressure is sent to the pneumatic actuator, so that the bending operation may be performed. FIG. 16A is a schematic sectional view of a tip bending portion of a bending-insertion assisting instrument that is formed of a pneumatic actuator, and FIG. 16B is a schematic cross-sectional view of the tip bending portion.

Cell lines 133U, 133D, 133R, and 133L where a plurality of hollow cells 131 is arranged in the axial direction of the tip bending portion 19, which is formed of a pneumatic actuator, are disposed in a total of four lines at peripheral positions that are different in the horizontal and vertical directions shown in FIG. 16B. An air pressure supply controller (not shown) is connected to each of the cell lines.

For example, if the tip bending portion 19 is intended to be bent down in the drawing, as shown in FIGS. 17A and 17B, the pressure of the respective hollow cells 131 of the cell line 133U is controlled to be increased by the air supplied from the air pressure supply controller, and the pressure of the cell line 133D is controlled to be decreased. Likewise, even if the tip bending portion 19 is intended to be bent in other directions, the pressure of the cell lines (133U and 133D, or 133R and 133L) facing each other may be controlled to be increased or decreased.

Meanwhile, air pressure is used as a driving source for the above-mentioned bending driving, but liquid pressure may also be used for the bending driving.

Further, the tip bending portion 19 may be made of a shape memory alloy and the shape memory alloy may be heated by an electric signal or the like in order to perform a bending operation.

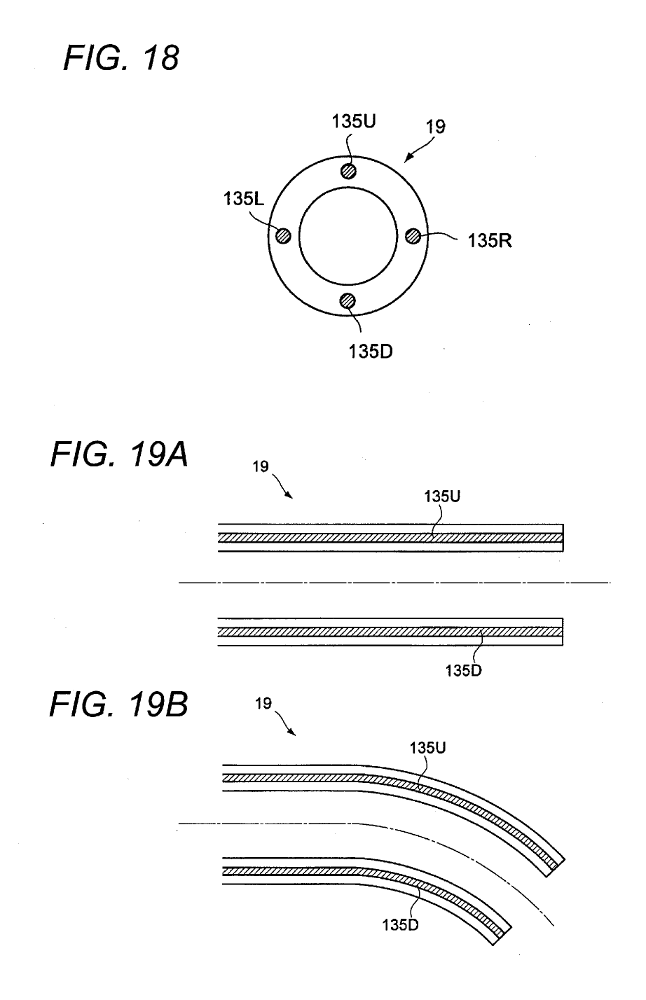

FIG. 18 is a schematic sectional view of a tip bending portion of a bending-insertion assisting instrument where a shape memory alloy is partially disposed, FIG. 19A is a schematic sectional view showing a linear state of the tip bending portion shown in FIG. 18, and FIG. 19B is a schematic sectional view showing a bent state of the tip bending portion.

As shown in FIG. 18, four bending members 135U, 135D, 135R, and 135L including a shape memory alloy are disposed in the tip bending portion 19 of the bending-insertion assisting instrument at peripheral positions that are different in the horizontal and vertical directions. Each of the bending members 135U, 135D, 135R, and 135L is disposed in the axial direction of the tip bending portion 19 as shown in FIG. 19A. A heating controller (not shown) is connected to each of the bending members.

Meanwhile, a Ni—Ti alloy, a Cu—Zn—Al alloy, a Fe—Mn—Si alloy, and the like may be used as the shape memory alloy. A well-known material such as a shape memory resin may be used other than a shape memory alloy.

For example, if the tip bending portion 19 is intended to be bent down in the drawing, as shown in FIG. 19B, an electric signal is supplied to the bending member 135D from the heating controller so that the bending member 135D is heated. Each of the bending members has a function of elongating and contracting in the longitudinal direction when being heated. In the example shown in FIG. 19B, the bending member 135D contracts in the longitudinal direction. Accordingly, the tip bending portion 19 is bent down as a whole.

If the structure using the above-mentioned pneumatic actuator or shape memory alloy is employed, it may be possible to obtain a structure where the operation of the tip bending portion 19 does not affect the intermediate extension portion 17 (see FIG. 2) at all.

As described above, the invention is not limited to the above-mentioned embodiment. Further, modifications and applications, which are to be performed by those skilled in the art on the basis of the description of the specification and known techniques, are also anticipated by the invention and are included in the range to be protected.

As described above, the following is disclosed in this specification.

(1) A bending-insertion assisting instrument bends an insertion path of a medical instrument being to be inserted into the body cavity in a desired shape so that the medical instrument is guided in the body cavity. The bending-insertion assisting instrument includes a tip bending portion, an intermediate extension portion, a bending operation portion and a shape switching operation portion. The tip bending portion includes a bendable bending mechanism. The intermediate extension portion has a long shape and is connected to the tip bending portion. The bending operation portion is disposed at a base end of the intermediate extension portion and bends the tip bending portion. The intermediate extension portion includes a shape retaining mechanism which selectively switches a state of the intermediate extension portion to a variable-shape state where the bent shape of the intermediate extension portion is freely changed and a shape retaining state where the bent shape of the intermediate extension portion is retained. The shape switching operation portion switches the respective states of the intermediate extension portion.

According to the bending-insertion assisting instrument, even though an insertion path along which various pieces of medical equipment are inserted into the body cavity has a complicated shape, it may be possible to make the various pieces medical equipment reliably follow the bent shape of the insertion path and to always accurately maintain the insertion path of the various medical equipment in a desired shape.

(2) The bending-insertion assisting instrument according to (1), the shape retaining mechanism is formed over the entire length of the intermediate extension portion.

According to the bending-insertion assisting instrument, it may be possible to maintain the intermediate extension portion, which is bent, over a long distance.

(3) The bending-insertion assisting instrument according to (1) or (2), the tip bending portion is bent in a plurality of directions at the same time.

According to the bending-insertion assisting instrument, it may be possible to bend the bending-insertion assisting instrument in different directions at the same time by a single operation, and to easily cope with a complicated bent shape.

(4) The bending-insertion assisting instrument according to any one of (1) to (3), the shape retaining mechanism includes a hollow space and protrusions. The hollow space introduces fluid between an inner wall and an outer wall of the intermediate extension portion. The protrusions are formed on the inner wall and the outer wall of the hollow space so as to face each other. The protrusions are engaged with each other when the fluid of the hollow space is discharged.

According to the bending-insertion assisting instrument, it may be possible to easily and selectively switch the state of the intermediate extension portion into a variable-shape state and a shape retaining state simply by supplying liquid to the hollow space or discharging liquid from the hollow space.

(5) The bending-insertion assisting instrument according to any one of (1) to (3), the shape retaining mechanism includes a plurality of short cylindrical joint rings and operation wires. The plurality of short cylindrical joint rings is connected in series. Respective adjacent joint rings of the plurality of joint rings are connected to each other by connecting shafts so as to be rotated relative to each other. The operation wires are provided parallel to a connecting direction of the joint rings.

According to the bending-insertion assisting instrument, it may be possible to bend the bending-insertion assisting instrument in an arbitrary direction and maintain the bending-insertion assisting instrument in a bent state by pulling or fixing the operation wire, and to change the bent state of the bending-insertion assisting instrument by loosening the operation wire.

(6) An insertion path securing apparatus includes the bending-insertion assisting instrument according to any one of (1) to (5) and a sheath tube. The sheath tube includes a first lumen where the tip bending portion and the intermediate extension portion of the bending-insertion assisting instrument are inserted. The sheath tube includes a tube-like insertion portion and an operation portion. The tube-like insertion portion has a long shape and is to be inserted into the body cavity. The operation portion is disposed at a base end of the tube-like insertion portion. The tube-like insertion portion includes a shape retaining mechanism which selectively switches a state of the tube-like insertion portion to a variable-shape state where the bent shape of the tube-like insertion portion is freely changed and a shape retaining state where the bent shape of the tube-like insertion portion is retained. The operation portion switches the respective states of the tube-like insertion portion.

According to the insertion path securing apparatus, it may be possible to secure a bent insertion path by inserting the insertion path securing apparatus into the body cavity so that the insertion path securing apparatus corresponds to a complicated bent shape.

(7) The insertion path securing apparatus according to (6), the insertion path securing apparatus has a function of repeatedly performing operations in order. The operations include (i) bending the tip bending portion of the bending-insertion assisting instrument and making the bending-insertion assisting instrument enter the shape retaining state; (ii) making the sheath tube enter the variable-shape state, moving a distal end of the sheath tube to a tip of the tip bending portion, and transferring the bent shape of the bending operation assisting instrument to the sheath tube; (iii) making the sheath tube enter the shape retaining state and retaining the bent shape of the bending operation assisting instrument on the sheath tube; and (iv) making the bending-insertion assisting instrument enter the variable-shape state and making at least the tip bending portion of the bending-insertion assisting instrument protrude from the first lumen.

According to the insertion path securing apparatus, it may be possible to insert the insertion path securing apparatus into the body cavity while bending the insertion path securing apparatus in an arbitrary shape, and to reliably secure a bent insertion path.

(8) The insertion path securing apparatus according to (6), the insertion path securing apparatus has a function of repeatedly performing operations in order. The operations include: (i) making the sheath tube enter the variable-shape state and moving a distal end of the sheath tube to a tip of the tip bending portion; (ii) bending the tip bending portion of the bending-insertion assisting instrument, making the bending-insertion assisting instrument enter the shape retaining state, and transferring the bent shape of the bending operation assisting instrument to the sheath tube; (iii) making the sheath tube enter the shape retaining state and retaining the bent shape of the bending operation assisting instrument on the sheath tube; (iv) making the bending-insertion assisting instrument enter the variable-shape state and making at least the tip bending portion of the bending-insertion assisting instrument protrude from the first lumen; and (v) making the bending-insertion assisting instrument enter the shape retaining state.

According to the insertion path securing apparatus, it may be possible to insert the insertion path securing apparatus into the body cavity while bending the insertion path securing apparatus in an arbitrary shape.

(9) The insertion path securing apparatus according to any one of (6) to (8), a second lumen is formed in the sheath tube. An endoscopic insertion portion being inserted into the body cavity is inserted into the second lumen.

According to the insertion path securing apparatus, it may be possible to operate the insertion path securing apparatus while making an observation with an endoscope, while ensuring a reliable operation.

(10) The insertion path securing apparatus according to any one of (6) to (8), a forceps hole of an endoscopic insertion portion is used as the first lumen of the sheath tube. The endoscopic insertion portion is inserted into the body cavity. The bending-insertion assisting instrument is inserted into the forceps hole.

According to the insertion path securing apparatus, it may be possible to easily secure an insertion path by using the endoscopic insertion portion of the endoscope as a sheath tube and operating the endoscopic insertion portion while combining the endoscopic insertion portion and the bending-insertion assisting instrument.

(11) A bending-insertion assisting instrument includes a tip bending portion, an intermediate extension portion and a device operating portion. The tip bending portion includes a bendable bending mechanism. The intermediate extension portion has a long shape and is connected to a base end of the tip bending portion. The intermediate extension portion includes a shape retaining mechanism which selectively switches a state of the intermediate extension portion to a variable-shape state where the bent shape of the intermediate extension portion is freely changed and a shape retaining state where the bent shape of the intermediate extension portion is retained. The device operating portion includes a bending operation portion and a shape switching operation portion. The bending operation portion is disposed at a base end of the intermediate extension portion and performs an operation for bending the tip bending portion. The shape switching operation portion switches the respective states of the intermediate extension portion. The bending operation portion and the shape switching operation portion are independently operated. The bending operation portion performs the operation for bending the tip bending portion without changing the bent shape of the intermediate extension portion.

According to the bending-insertion assisting instrument, it may be possible to independently operate the bending operation portion and the shape switching operation portion and to improve the operability for an operator. Further, even though the bending operation portion is operated, the shape of the intermediate extension portion is not changed. Even when bent shapes are formed at a plurality of positions, it may be possible to continue to retain each bent shape as it is.

(12) The bending-insertion assisting instrument according to (11), the bending mechanism includes a plurality of joint rings which are connected in a longitudinal direction and traction wires. Tips of the traction wires are fixed to a joint ring which is positioned at a tip in the longitudinal direction. The intermediate extension portion includes a guide member which guides the traction wires on the central axis of the intermediate extension portion.

According to the bending-insertion assisting instrument, it may be possible to exert a stable shape retaining property without affecting the shape of the intermediate extension portion even though tension is generated on the traction wire by an operation for bending the tip bending portion.

(13) The bending-insertion assisting instrument according to (11), the bending mechanism includes a fluidic actuator.

According to the bending-insertion assisting instrument, it may be possible to obtain the structure where an operation for bending the tip bending portion does not affect the intermediate extension portion at all.

(14) The bending-insertion assisting instrument according to (11), the bending mechanism is made of a shape memory alloy.

According to the bending-insertion assisting instrument, it may be possible to obtain the structure where an operation for bending the tip bending portion does not affect the intermediate extension portion at all.

There is an insertion path securing method of guiding an insertion instrument which is to be inserted into the body cavity by using an insertion path securing apparatus. The insertion path securing apparatus includes a bending-insertion assisting instrument and a sheath tube which includes a first lumen where an intermediate extension portion and a tip bending portion of the bending-insertion assisting instrument are inserted. The insertion path securing method includes: bending the tip bending portion of the bending-insertion assisting instrument and making the bending-insertion assisting instrument enter the shape retaining state; making the sheath tube enter the variable-shape state, moving a distal end of the sheath tube to a tip of the tip bending portion, and transferring the bent shape of the bending operation assisting instrument to the sheath tube; making the sheath tube enter the shape retaining state and retaining the bent shape of the bending operation assisting instrument at the sheath tube; and making the bending-insertion assisting instrument enter the variable-shape state and making at least the tip bending portion of the bending-insertion assisting instrument protrude from the first lumen. The steps of the insertion path securing method are performed in order repeatedly.

There is an insertion path securing method of guiding an insertion instrument which is to be inserted into the body cavity by using an insertion path securing apparatus. The insertion path securing apparatus includes a bending-insertion assisting instrument and a sheath tube which includes a first lumen where an intermediate extension portion and a tip bending portion of the bending-insertion assisting instrument are inserted. The insertion path securing method includes: making the sheath tube enter the variable-shape state and moving a distal end of the sheath tube to a tip of the tip bending portion; bending the tip bending portion of the bending-insertion assisting instrument, making the bending-insertion assisting instrument enter the shape retaining state, and transferring the bent shape of the bending operation assisting instrument to the sheath tube; making the sheath tube enter the shape retaining state and retaining the bent shape of the bending operation assisting instrument at the sheath tube; making the bending-insertion assisting instrument enter the variable-shape state and making at least the tip bending portion of the bending-insertion assisting instrument protrude from the first lumen; and making the bending-insertion assisting instrument enter the shape retaining state. The steps of the insertion path securing method are performed in order repeatedly.

What is claimed is:

1. A bending-insertion assisting instrument which bends an insertion path of a medical instrument being to be inserted into the body cavity in a desired shape so that the medical instrument is guided in the body cavity, the bending-insertion assisting instrument comprising:
a tip bending portion that includes a bendable bending mechanism;
an intermediate extension portion that has a long shape and that is connected to the tip bending portion;
a bending operation portion that is disposed at a base end of the intermediate extension portion and that bends the tip bending portion,
wherein the intermediate extension portion includes a shape retaining mechanism which selectively switches a state of the intermediate extension portion to a variable-shape state where the bent shape of the intermediate extension portion is freely changed and a shape retaining state where the bent shape of the intermediate extension portion is retained, and
a shape switching operation portion that switches the respective states of the intermediate extension portion.

2. The bending-insertion assisting instrument according to claim 1,
wherein the shape retaining mechanism is formed over the entire length of the intermediate extension portion.

3. The bending-insertion assisting instrument according to claim 1,
wherein the tip bending portion is bent in a plurality of directions at the same time.

4. The bending-insertion assisting instrument according to claim 1,
wherein the shape retaining mechanism includes;
a hollow space which introduces fluid between an inner wall and an outer wall of the intermediate extension portion, and
protrusions which are formed on the inner wall and the outer wall of the hollow space so as to face each other, and
the protrusions are engaged with each other when the fluid of the hollow space is discharged.

5. The bending-insertion assisting instrument according to claim 1,
wherein the shape retaining mechanism includes
a plurality of short cylindrical joint rings which is connected in series and of which respective adjacent joint rings are connected to each other by connecting shafts so as to be rotated relative to each other, and
operation wires which are provided parallel to a connecting direction of the joint rings.

6. An insertion path securing apparatus comprising:
the bending-insertion assisting instrument according to claim 1; and
a sheath tube that includes a first lumen where the tip bending portion and the intermediate extension portion of the bending-insertion assisting instrument are inserted,
wherein the sheath tube includes a tube-like insertion portion and an operation portion, the tube-like insertion portion which has a long shape and which is to be inserted into the body cavity, the operation portion which is disposed at a base end of the tube-like insertion portion,
the tube-like insertion portion includes a shape retaining mechanism which selectively switches a state of the tube-like insertion portion to a variable-shape state where the bent shape of the tube-like insertion portion is freely changed and a shape retaining state where the bent shape of the tube-like insertion portion is retained, and
the operation portion switches the respective states of the tube-like insertion portion.

7. The insertion path securing apparatus according to claim 6,
wherein the insertion path securing apparatus has a function of repeatedly performing operations in order, the operations include: (i) bending the tip bending portion of the bending-insertion assisting instrument and making the bending-insertion assisting instrument enter the shape retaining state; (ii) making the sheath tube enter the variable-shape state, moving a distal end of the sheath tube to a tip of the tip bending portion, and transferring the bent shape of the bending operation assisting instrument to the sheath tube; (iii) making the sheath tube enter the shape retaining state and retaining the bent shape of the bending operation assisting instrument on the sheath tube; and (iv) making the bending-insertion assisting instrument enter the variable-shape state and making at least the tip bending portion of the bending-insertion assisting instrument protrude from the first lumen.

8. The insertion path securing apparatus according to claim 6,
wherein the insertion path securing apparatus has a function of repeatedly performing operations in order, the operations include: (i) making the sheath tube enter the variable-shape state and moving a distal end of the sheath tube to a tip of the tip bending portion; (ii) bending the tip bending portion of the bending-insertion assisting instrument, making the bending-insertion assisting instrument enter the shape retaining state, and transferring the bent shape of the bending operation assisting instrument to the sheath tube; (iii) making the sheath tube enter the shape retaining state and retaining the bent shape of the bending operation assisting instrument on the sheath tube; (iv) making the bending-insertion assisting instrument enter the variable-shape state and making at least the tip bending portion of the bending-insertion assisting instrument protrude from the first lumen; and (v) making the bending-insertion assisting instrument enter the shape retaining state.

9. The insertion path securing apparatus according to claim 6,
wherein a second lumen is formed in the sheath tube, the second lumen into which an endoscopic insertion portion being inserted into the body cavity is inserted.

10. The insertion path securing apparatus according to claim 6,
wherein a forceps hole of an endoscopic insertion portion is used as the first lumen of the sheath tube, the endoscopic insertion portion being inserted into the body cavity, and
the bending-insertion assisting instrument is inserted into the forceps hole.

11. A bending-insertion assisting instrument comprising:
a tip bending portion that includes a bendable bending mechanism;
an intermediate extension portion that has a long shape, that is connected to a base end of the tip bending portion, and that includes a shape retaining mechanism, the shape retaining mechanism which selectively switches a state of the intermediate extension portion to a variable-shape state where the bent shape of the intermediate extension portion is freely changed and a shape retaining state where the bent shape of the intermediate extension portion is retained; and
a device operating portion that includes a bending operation portion and a shape switching operation portion, the bending operation portion being disposed at a base end of the intermediate extension portion and performing an operation for bending the tip bending portion, and the shape switching operation portion switching the respective states of the intermediate extension portion,
wherein the bending operation portion and the shape switching operation portion are independently operated, and
the bending operation portion performs the operation for bending the tip bending portion without changing the bent shape of the intermediate extension portion.

12. The bending-insertion assisting instrument according to claim 11,
wherein the bending mechanism includes a plurality of joint rings which are connected in a longitudinal direction and traction wires,
tips of the traction wires are fixed to a joint ring which is positioned at a tip in the longitudinal direction, and
the intermediate extension portion includes a guide member which guides the traction wires on the central axis of the intermediate extension portion.

13. The bending-insertion assisting instrument according to claim 11,
wherein the bending mechanism includes a fluidic actuator.

14. The bending-insertion assisting instrument according to claim 11,
wherein the bending mechanism is made of a shape memory alloy.

15. An insertion path securing method for guiding an insertion instrument which is to be inserted into the body cavity by using an insertion path securing apparatus, the insertion path securing apparatus including a bending-insertion assisting instrument and a sheath tube which includes a first lumen where an intermediate extension portion and a tip bending portion of the bending-insertion assisting instrument are inserted, the insertion path securing method comprising:
bending the tip bending portion of the bending-insertion assisting instrument and making the bending-insertion assisting instrument enter the shape retaining state;
making the sheath tube enter the variable-shape state, moving a distal end of the sheath tube to a tip of the tip bending portion, and transferring the bent shape of the bending operation assisting instrument to the sheath tube;
making the sheath tube enter the shape retaining state and retaining the bent shape of the bending operation assisting instrument at the sheath tube; and
making the bending-insertion assisting instrument enter the variable-shape state and making at least the tip bending portion of the bending-insertion assisting instrument protrude from the first lumen, and
wherein the steps of the insertion path securing method are performed in order repeatedly.

16. An insertion path securing method for guiding an insertion instrument which is to be inserted into the body cavity by using an insertion path securing apparatus, the insertion path securing apparatus including a bending-insertion assisting instrument and a sheath tube which includes a first lumen where an intermediate extension portion and a tip bending portion of the bending-insertion assisting instrument are inserted, the insertion path securing method comprising:
making the sheath tube enter the variable-shape state and moving a distal end of the sheath tube to a tip of the tip bending portion;
bending the tip bending portion of the bending-insertion assisting instrument, making the bending-insertion assisting instrument enter the shape retaining state, and transferring the bent shape of the bending operation assisting instrument to the sheath tube;
making the sheath tube enter the shape retaining state and retaining the bent shape of the bending operation assisting instrument at the sheath tube;
making the bending-insertion assisting instrument enter the variable-shape state and making at least the tip bending portion of the bending-insertion assisting instrument protrude from the first lumen; and
making the bending-insertion assisting instrument enter the shape retaining state, and
wherein the steps of the insertion path securing method are performed in order repeatedly.
